(19) United States
(12) Patent Application Publication
(10) Pub. No.: US 2025/0253025 A1
(43) Pub. Date: Aug. 7, 2025

(54) Mobile Application for Treating Childhood Obesity

(71) Applicant: Eleonor April Jones, Austin, TX (US)

(72) Inventor: Eleonor April Jones, Austin, TX (US)

(21) Appl. No.: 18/916,227

(22) Filed: Oct. 15, 2024

Related U.S. Application Data
(60) Provisional application No. 63/549,811, filed on Feb. 5, 2024.

(51) Int. Cl. G16H 20/30; A63F 13/46; G06F 3/0484; G16H 10/60; G16H 20/60

(52) U.S. Cl. CPC G16H 20/30; A63F 13/46; G06F 3/0484; G16H 10/60; G16H 20/60

(57) ABSTRACT

The invention is a mobile application to treat and prevent childhood obesity by collecting a user's entering the health information. Providing daily gamified lessons, nutritional information, exercise tips, links to social media offering continued education on weight loss and fitness. The invention is adapted to the audience by gamification. Gamifying promotes user engagement and motivation by rewards through a point system where points are earned by completing quests. The quests are designed to educate and promote positive behaviors.

### CROSS REFERENCE TO RELATED APPLICATIONS

This application claims priority to co-pending U.S. Provisional Application, Serial No. 63/549,811, filed on Feb. 5, 2024, which is hereby incorporated by reference for all purposes.

### BACKGROUND

The invention is a mobile application used to treat childhood obesity. The invention addresses three problems in the treatment of childhood obesity: (1) the patient's problem of poor eating habits, poor physical health habits, the lack of a trained professional accountability partner to develop personalized treatment plans and to go through the treatment plan side by side with the patient, the lack of follow up, and poor current solutions to education and awareness. Patients often feel alone when going through the journey. (2) The provider problem is that there is no scalable solution to patient follow-up and ensuring compliance with the treatment plan, no scalable solution for providing personalized follow-up and accountability. Additionally, there is no solution to solving the patient feelings of being alone on the journey. Also, the provider lacks quantity and quality of on-demand real-time data for review and follow-up. (3) An execution problem: most treatment plans and programs are built on old delivery models, utilize antiquated technology for delivery, and don't incorporate modern neuroscience and psychology practices to enable children to create and maintain healthy habits. The invention facilitates the use of modern neuroscience and psychology practices for implementation in the treatment plan.

Most currently recommended applications designed for managing childhood obesity predominantly adopt the gamified approach, featuring functionalities such as calorie counting and exercise tracking to sustain the engagement of young patients. However, a notable deficiency exists in personalization, and these applications do not precisely align with the nuanced requirements for managing a chronic condition like obesity. Compelling research underscores the adverse impact of calorie-counting apps, revealing a propensity for fostering long-term disordered eating behaviors among teenagers. Notably, the absence of applications that adeptly traverse the digital landscape where young individuals reside and assimilate information is evident. Moreover, a discernible gap persists in adherence to the recently issued guidelines by the American Academy of Pediatrics (AAP), as no existing applications currently align with these recommendations.

For example, some applications are geared to help healthcare providers and parents learn to talk to their kids/users about obesity but do not provide actual interaction with the user. Other applications focus on adult-based psychology but do not consider the barriers AAP recognizes as most significant in the pediatric population. Again, no connection between the provider/user is provided. Moreover, the psychology is very generalized, and information is basic and gives the same psychology to everyone; it does not help differentiate the eating habits of each individual and doesn't provide reassessment over time. Lastly, other applications focus on weight, calorie counting, and food labeling. When this kind of behavior is directed towards children, however, it can lead to disordered eating/or eating disorders.

In sum, there is a distinct need for more tailored, personalized, and guideline-adherent digital solutions in childhood obesity management. How the invention meets the needs of treating childhood obesity is discussed below.

### SUMMARY

The invention is a mobile application that treats and prevents childhood obesity by collecting a user's health information. Providing daily assessments in the form of gamified lessons, nutritional information, exercise tips, social media and other web based links offering continued weight loss and fitness education. The invention is adapted to the audience by gamification. Gamifying promotes user engagement and motivation by rewards through a point system where points are earned by completing quests. The quests are designed to educate and encourage positive behaviors.

The invention customizes the treatment plan by running a barrier assessment. Analyzing the barrier assessment and producing a prioritized list goals based on the barrier assessment. Gamifying the goals by converting the goals into quests. Where the parameters of the quest are defined as a SMART goal (to be described later).

### BRIEF DESCRIPTION OF THE DRAWINGS

FIG. 1 shows the dashboard.

FIG. 2 is an example of a daily quest in the form of a multiple-choice question.

FIG. 3 is an example of a correct answer to a multiple-choice question.

FIG. 4 is an example of a daily quest in the form of a lesson.

FIG. 5 is an example of a virtual device.

FIG. 6. Is an example of a journal entry.

FIG. 7 is an example of a journal entry in the form of a question.

FIG. 8 is an example of a journal entry in the form of an emoji.

FIG. 9 is an example of a journal entry in the form of a summary.

FIG. 10 is a block diagram of the elements of the App.

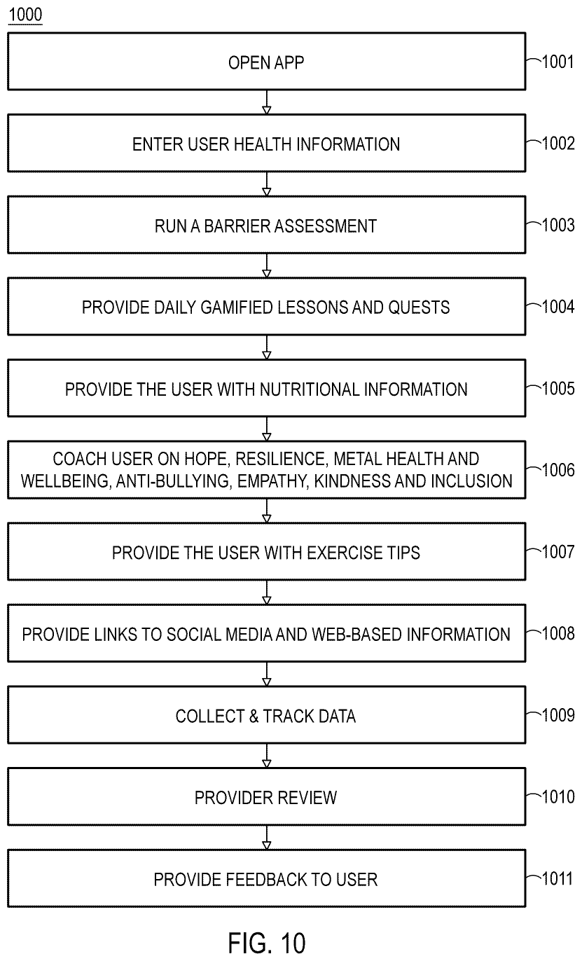

### DETAILED DESCRIPTION

The invention will become better understood by reviewing the following detailed description in conjunction with the figures. The detailed description and figures provide merely examples of the various inventions described herein. Those skilled in the art will understand that the disclosed examples may be varied, modified, and altered without departing from the scope of the inventions described herein. Many variations are contemplated for different applications and design considerations; however, for the sake of brevity, each and every contemplated variation is not individually described in the following detailed description.

### Detailed Description

Throughout the following detailed description, a variety of examples are provided. Related features in the examples may be identical, similar, or dissimilar in different examples. For the sake of brevity, related features will not be redundantly explained in each example. Instead, using related feature names will cue the reader that the feature with a related name may be similar to the related feature in an example explained previously. Features specific to a given example will be described in that particular example. The reader should understand that a given feature need not be the same or similar to the specific portrayal of a related feature in any given figure or example.

### Mobile Application for Treating Childhood Obesity

The MOBILE APPLICATION FOR TREATING CHILDHOOD OBESITY will now be described. The invention is a mobile application positioned to serve as an educational platform, imparting essential skills regarding obesity to children and their families. By breaking down communication barriers that may have previously hindered practical discussions on weight-related issues, the invention seeks to facilitate a more open and constructive dialogue within families. This, in turn, is anticipated to contribute to overcoming obstacles that impede successful weight loss efforts. Beyond immediate interventions, the invention is designed to equip children with the knowledge and tools necessary to adopt healthy lifestyle modifications and, crucially, to sustain these changes throughout their lifetimes. This holistic approach aims not only to address the immediate health concerns of the users but also to instill enduring habits that promote long-term well-being.

The invention serves as a conduit for disseminating pertinent information to children greater than 6-years old within the digital realm by aligning with their preferred channels for information consumption. The invention facilitates a seamless and targeted content delivery by strategically occupying the digital space where this demographic routinely accesses and absorbs information. Optionally, it can be valuable tool for healthcare providers, enabling them to gather essential data critical for advancing medical understanding and enhancing treatment modalities. Through this dual functionality, the application caters to the informational needs of its users and contributes to the ongoing progress and innovation within the healthcare field. For the purposes of this invention non-limiting examples of a provider include: medical doctors, nutritionists, therapists, psychologists, athletic coaches, wellness coaches, a public or private school program, businesses in the health and weight loss industry (with non-limiting examples such as Jenny Craig, Noom, Atkins, or Weight Watchers), programs provided by health insurers, the user's family or a designated third party.

The invention is the first mobile application built to seamlessly integrate into children's digital and physical daily lives to establish and maintain physical, mental, and emotional health habits targeted toward solving childhood obesity. The invention is a tool to solve childhood obesity by targeting three areas: physical technology, humans, AI resources, and the social stigma of obesity. The invention utilizes innovative education and awareness modalities specifically designed for children's digital lives in the modern world. The invention captures data, tracks behavior, and monitors active research. Utilizing the former information by the user and provider gives a means to meet all the suggested requirements recommended by AAP. Moreover, the invention reduces the limitations in healthcare, such as the lack of resources and rising costs of healthcare visits, all while increasing the amount of contact between the user and provider.

The invention comprises three primary components, each strategically designed to fulfill distinct roles in addressing childhood obesity. The first component is the user/user view, featuring a personalized user log-in system. This interface facilitates daily gamified lessons, providing users with nutritional and exercise tips for holistic well-being. Moreover, integrated links to social media platforms or web platforms that offer a dynamic avenue for continued education, presented in pictorial, written, or video format. The gamification aspect provides a point-scoring system, wherein users garner points for interaction with the game. Optionally additional points can be earned through likes, shares, and posts on the linked social media, thereby incentivizing ongoing engagement with the application. The second component is the provider view, tailored for the provider tasked with tracking and collecting pertinent data. Initially, this data monitors users' adherence to recommendations and lifestyle changes, enabling providers to assess scoring metrics without necessitating immediate follow-up. Over time, the accumulated data assumes a dual role, ensuring user compliance and contributing to broader research endeavors within the realm of childhood obesity. The third and final component involves the parent, where the application extends its influence beyond the individual user. Parents or legal guardians actively engage with the mobile platform, fostering a collaborative approach to addressing childhood obesity. This interactive space allows parents to connect with the user, leveraging social media links to create menus and compile grocery lists. By promoting community building and nurturing an environment conducive to success, this parental component enhances the holistic support structure surrounding the user, maximizing the potential for positive outcomes in the journey towards healthier living.

The invention employs a digitally driven learning modality via a web/mobile application integrating modern neuroscience and behavioral psychology. The invention will teach children to set goals and remind them daily that they are worth the effort. It will track their motivation, well-being, sleep, water intake, and daily check-ins moving the user toward behavioral modifications. The digitally driven strategy provides education and awareness via push notifications through the mobile application, social media, or an influencer strategy. Auto-populated reminders will be sent at a frequency determined by the user. In one non-limiting example, if the user doesn't check in or score 60% or lower over 7 days on the daily check-in, the provider will be flagged to contact the user or provider directly to follow up or, optionally, to schedule a face-to-face appointment. Integrating into these three digital places where children frequent will decrease the stigma many users feel. Knowing that the provider is checking in on the user will remove the weight stigma that many users feel when they work with a provider after a diagnosis of obesity.

The application is preloaded with intake forms, daily gamified lessons, and dietary and exercise information. There are also preloaded links to web or website based educational resources also including information found on social media platforms, with non-limiting examples being Instagram, TikTok, Facebook, and X. The user will download the App onto a mobile device and complete the intake forms on their own, with a parent, a third party, or at a clinic. This data is stored in electronic form with non-limiting examples including an electronic form service like Jotform, on the App, on a phone, tablet or other device, or a cloud-based service. The stored data can be transferred to an electronic document such as spreadsheet or a text document, with non-liming examples being Excel spreadsheet, Google Sheets, MS Word, or MS Power Point. The provider will review and categorize the participants' scores for further evaluation. In addition, the intake forms will be assessed for statistical significance. The provider will reassess the intake forms every 3 months.

The participants will also be able to complete daily gamified lessons, which monitor and track participant compliance. The gamified lessons are a series of questions assessing participant compliance with lifestyle adjustments. An assessment will question participants' continued motivation, daily activity level, food variation, and ability to avoid excess over the day. This differs from the calorie-counting and calorie-burning mobile applications that are currently available for the child who is obese. An assessment score will be sent to the electronic form service and stored for further evaluation by the provider. The provider will also review and categorize all the information to evaluate for compliance. The provider will share the feedback with participants via email once a week. The second daily assessment will assess the participants' sleep, water intake, and overall feelings of daily wellness. The assessment will be scored, sent to the electronic form service, and stored for further evaluation by the provider. The provider will review and categorize all the information to evaluate compliance. The provider will use the information to create feedback, which the provider will share with participants via email once a week. The participants' utilization of social media platforms during treatment will be systematically monitored through metrics such as likes, posts, and tags on their linked accounts. The overseeing provider thoroughly examines integrated social media accounts within the mobile application, focusing on the interactions of the user/user. This monitoring process serves the dual purpose of assessing both quantitative and qualitative aspects of engagement.

Further, the treatment will be gamified using judiciously allocated reward points that acknowledge the nuanced nature of participants' contributions within a structured reward/praise matrix. The points serve as tangible recognition, intricately tied to the observed interactions, creating a comprehensive approach to incentivizing and acknowledging participants' active and meaningful involvement in the treatment. The hope is that the invention will automate all of the portions the provider does on the back end with the tracking, scoring, and categorizing.

In one embodiment, the invention is embedded in a pediatric obesity clinic. The application will utilize medically based gamified lessons delivered digitally through the mobile application. These gamified lessons aim to determine barriers to weight loss, including physical activity level, eating patterns, nutritional deficits, or social/community/physical environment barriers, and create an individualized wellness plan. The wellness plan will include appropriate nutritional tips and exercise prescriptions that will be monitored and updated every 2-3 months or as needed. AI software/algorithm developments will create scalable individualized treatment plans across large groups.

In one non-limiting example, the health assessment can be completed using a survey that asks questions about mental health, nutrition, and physical activity. A non-limiting example of the format of a question for mental health is, “How often do you find it hard to choose healthy snacks when you feel upset or stressed?” Similarly, for nutrition, “How often do you drink sugary drinks like soda or juice?” and for physical activity, “How often do you choose active play (like tag or hide-and-seek) instead of video games?” The user then selects from a list of answers such as: “Never, Sometimes, Often, or Always.” The survey results are used to build a treatment plan.

The treatment plan comprises a series of goals using the SMART goal format. A SMART goal is Specific, Measurable, Attainable, Relevant, and Time Bound. A non-limiting example of a SMART goal for health is, “I will choose a healthy snack (like a piece of fruit or a handful of nuts) instead of an unhealthy one (like chips or candy) at least 3 days a week for the first 30 days, gradually increasing to 6 days a week by the end of the 60 days.” Similarly, for nutrition and physical activity, “I will replace one sugary drink with water every day, starting with 3 days a week for the first 30 days and increasing to 6 days a week by the end of the 60 days,” and “I will spend at least 15 minutes each day playing an active game like tag, hide-and-seek, or catch, starting with 3 days a week for the first 30 days and increasing to nearly daily by the end of the 60 days.” By reaching the goals, the user's health will improve. Gamification keeps the user engaged and motivated.

Gamification is a method that helps the user complete the treatment plan by making the process fun. It provides structured fun. Using the App., gamification reinforces education and correct behavior and encourages user engagement and motivation.

The App. includes a dashboard that provides entry to the App. providing a picture of the avatar, lists of daily quests, status of rewards, access to other functions such as access to functions such as the journal, and a list of SMART goals. Gamification motivates by converting education and SMART goals into fun activities. For example, a daily quest page shows the avatar in an entertaining virtual environment such as a den, beach, castle, etc. The avatar provides education by reading off the lesson for the day. Further education is provided by answering questions to a quiz. Engagement is encouraged by interaction with a journal. The journal offers a motivational quote for the day and promotes journal entries in a question-and-answer format, e.g., how much water did you drink today? Did you play outside today? Or did you eat your veggies today? Mental health is tracked using Emojis on a calendar.

A reward system is used for motivation. Non-limiting examples include tokens and an interactive Avatar. Each time a task is completed, a token is awarded. Tokens include winning coins, trophies, graphics showing winning streaks, etc. For example, after the daily lesson, tokens, such as a coin, can be awarded by engaging with a piece of animation, such as spinning a prize wheel that determines the number of coins awarded. Similarly, after completing a journaling task, coins are provided by pulling an animated lever. As multiple tasks are completed, earn higher level awards, such as trophies. Rewards are shown on the dashboard or stored in items consistent with the virtual environment, e.g., coins are stored in a treasure chest in a castle.

Further motivation is provided by engagement with an interactive avatar. Correct behavior is rewarded, and incorrect behavior is discouraged. Continued correct behavior enhances the avatar, while incorrect behavior diminishes the avatar.

For example, the user selects the interactive avatar, which can take any form, such as a dog, a cat, or a dragon. In this non-limiting example, the interactive avatar is a dragon. The dragon's virtual environment is a cave. The reward coins are stored in a sack. Proper completion of a goal causes the dragon to appear by hatching from an egg. Incorrect behavior may cause the dragon to go away. Proper behavior causes the dragon to return.

User engagement with the interactive avatar provides a way to use coins and trophies. For example, coins can be spent to enhance the interactive avatar. For example, ten coins give the dragon wings. If the dragon leaves, coins could be paid to bring the dragon back. The reward system and engagement with the interactive avatar provide motivation to earn tokens earned by proper behavior and accomplishing SMART goals. Which in turn moves the user through the treatment plan.

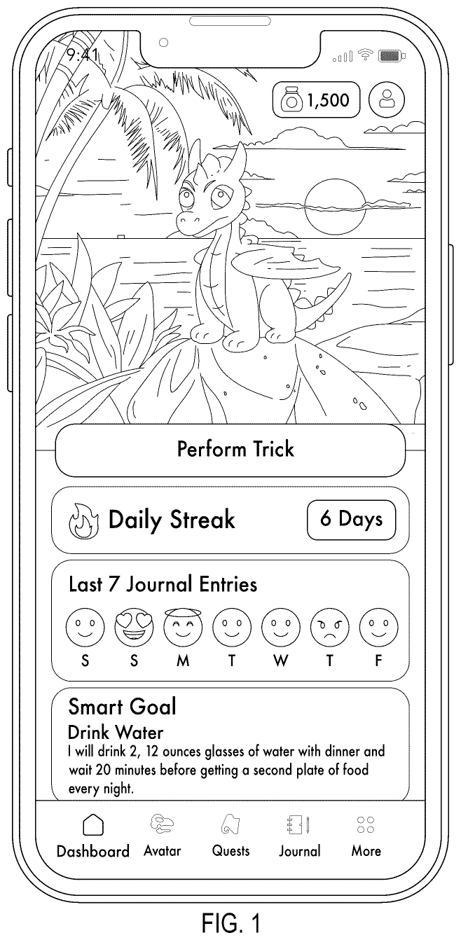

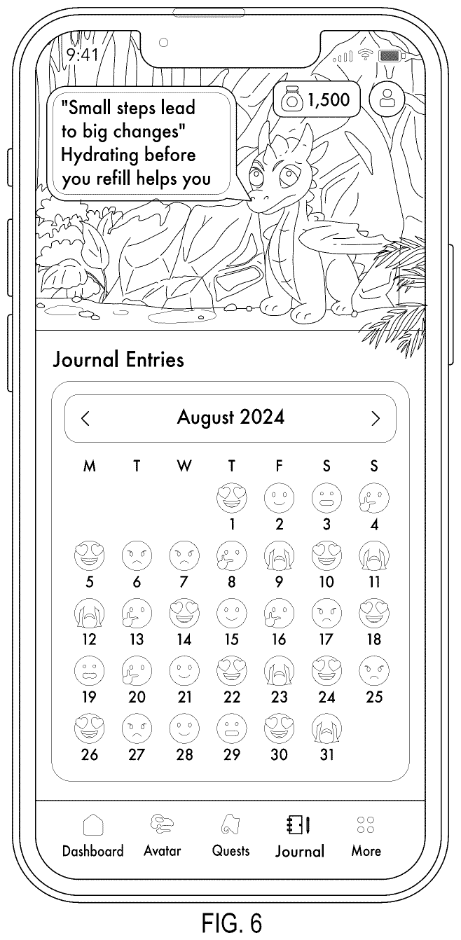

FIGS. 1-9 provide the following non-limiting examples: FIG. 1 presents the dashboard. It shows the avatar, provides feedback to the user, and promotes engagement. Engagement is encouraged by pressing a button that has the avatar perform a trick; the number of coins earned is also displayed. Feedback includes the “daily streak,” the quality of the journal entries for the week, and reminding the user of the SMART goal.

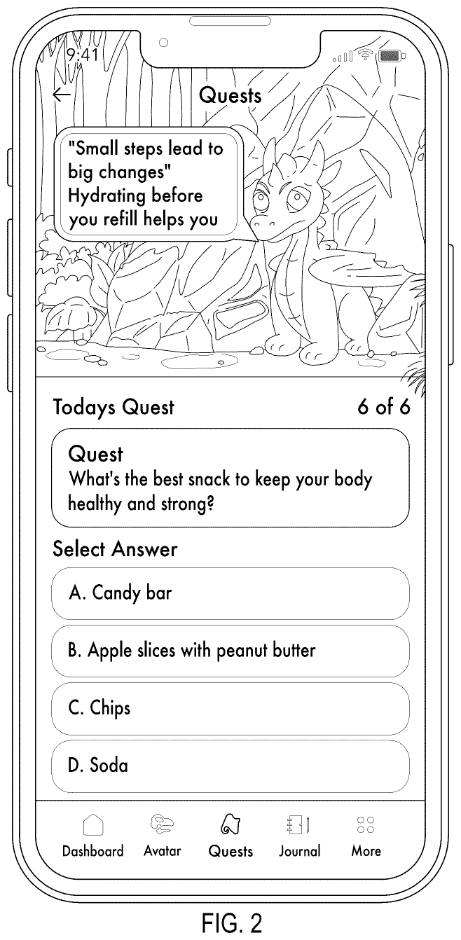

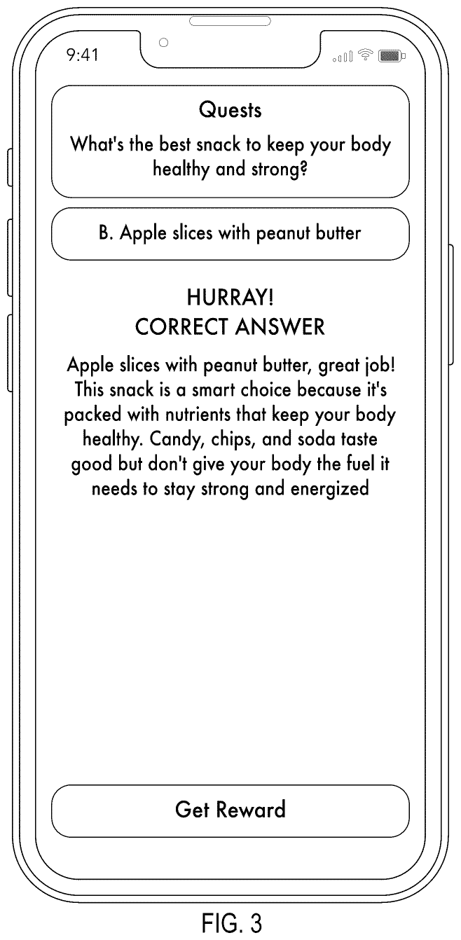

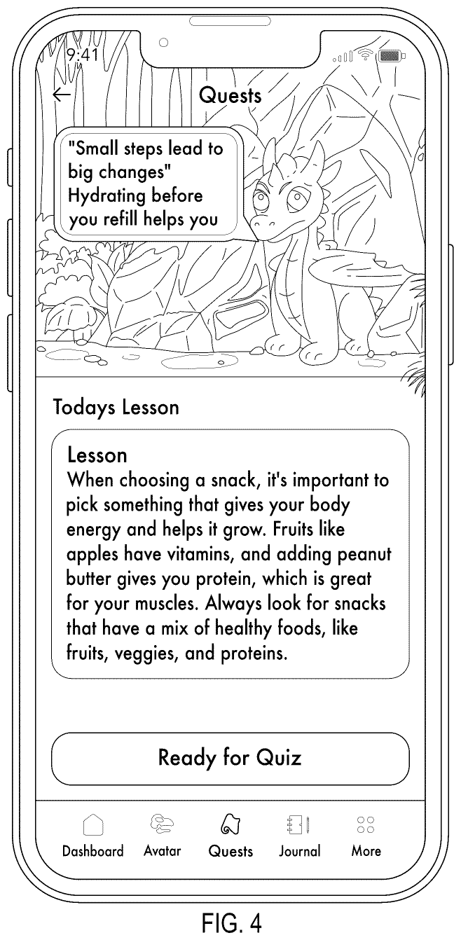

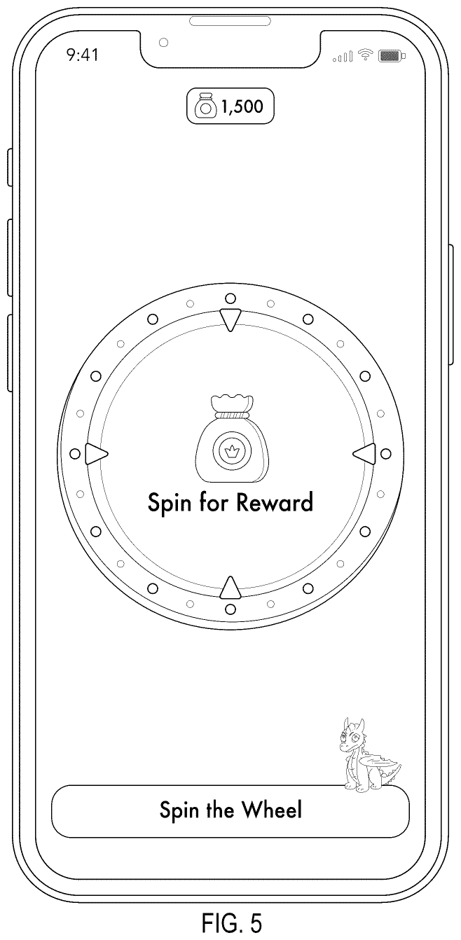

FIGS. 2-4 provide non-limiting examples of quests that educate the user. FIG. 2 shows the avatar interacting with the user by giving a tip in a speech bubble. FIG. 2 provides an example of a quest in the form of a multiple-choice question; FIG. 3 shows an example of the response to the correct answer for the question. Where FIG. 4 provides a non-limiting example of a quest in the form of a lesson. Completing quests allows the user to earn coins. Earning coins provides motivation and engagement. For example, FIG. 5 shows how to earn coins by spinning a wheel.

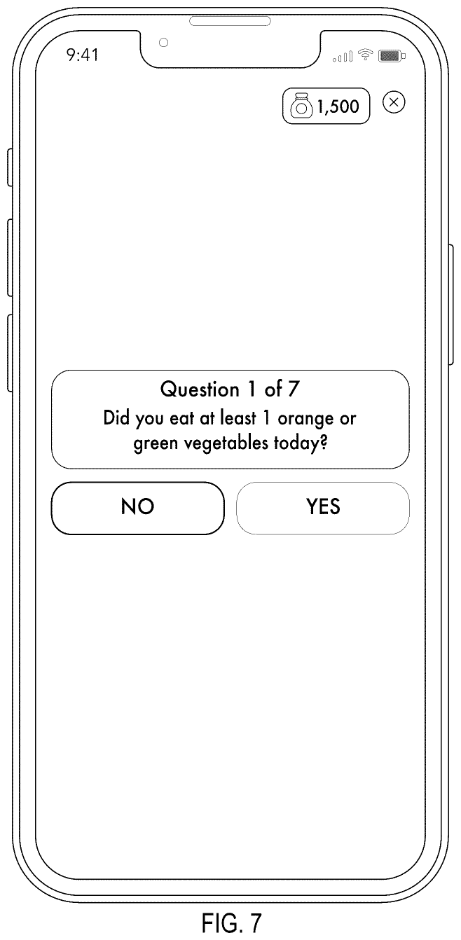

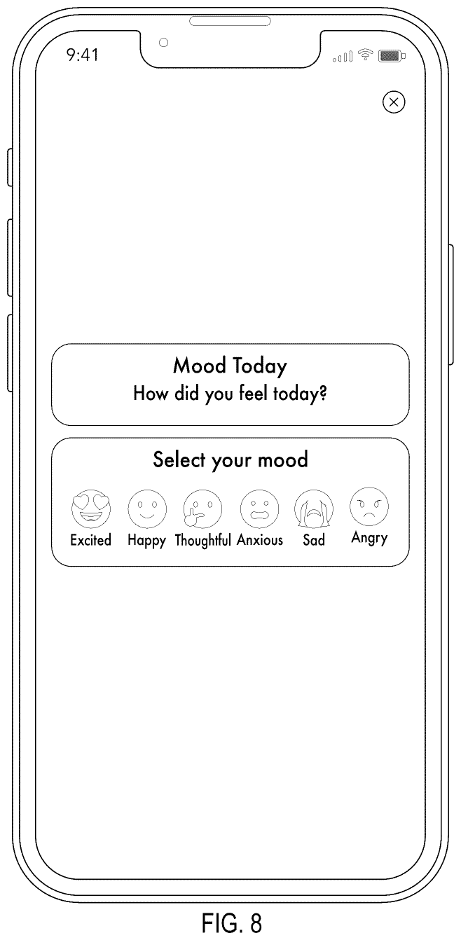

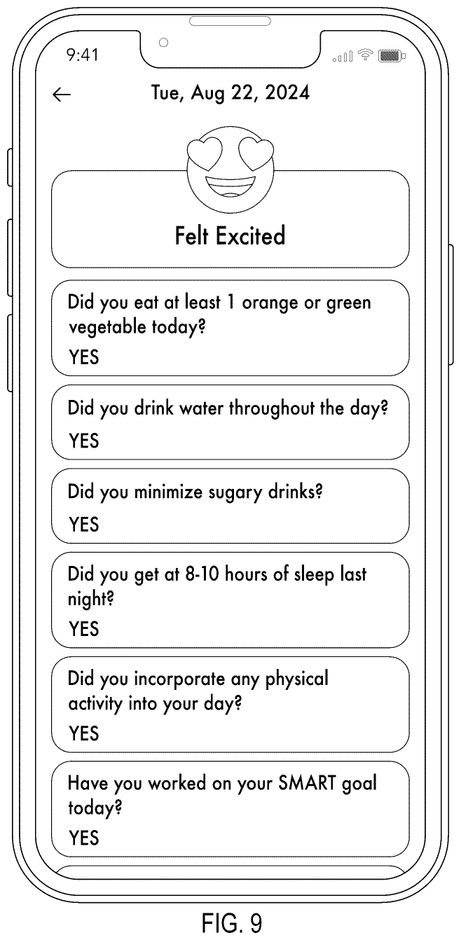

FIGS. 7-9 show non-limiting examples of journal entries. FIG. 7 makes a journal entry by answering a nutritional question. FIG. 8 inquires about the user's mood, and FIG. 9 provides a summary of journal entries.

Another embodiment of the App, hereinafter, Healthquest uses the following steps to address childhood obesity:

1. Removing Weight Stigma: HealthQuest is dedicated to promoting a positive, inclusive approach to health. The app focuses on overall well-being and holistic health, rather than just weight loss. By emphasizing mindfulness, intuitive eating, and physical activity, HealthQuest helps users build a healthy relationship with their bodies and self-image, free from judgment.

2. Integration of AI and Screening Tools: AI and specially designed screening tools assess users' barriers to a healthy lifestyle or weight loss. These tools prioritize challenges such as mental well-being, physical activity limitations, or nutrition concerns. The AI personalizes wellness plans based on individual needs, placing users on a suitable schedule, a non-limiting example is a 60-day wellness quest with check-ins every 15 days to adjust SMART goals. The AI considers users' age, learning style preferences, and specific challenges.

3. As a Comprehensive Wellness App: HealthQuest addresses various social determinants of health, including mental well-being/mindfulness, nutrition and intuitive eating, physical activity, and bullying. The app promotes healthy habits and emotional resilience, focusing on a holistic sense of wellness.

5. Focus on Mindfulness and Mental Well-being: The platform guides users through mindfulness practices and emotional regulation activities, fostering resilience, empathy, and self-confidence.

6. Addressing Physical Activity Limitations: HealthQuest offers accessible wellness plans for all physical activity levels, providing gradual improvements for less active users and challenging goals for those with higher activity levels.

7. Nutrition and Intuitive Eating: The HealthQuest emphasizes intuitive eating, encouraging users to listen to their bodies and adopt mindful eating habits, moving away from restrictive dieting.

8. Family Plan and Interaction: The family plan allows family members to complete quests together, interact, and build healthy habits collaboratively. Family check-ins every 15 days help adjust SMART goals for steady progress.

9. Bullying and Empathy: Lessons on bullying and social challenges promote empathy and positive social interactions. Mindfulness content equips users to handle difficult situations and build self-esteem.

10. Community Engagement and Nutrition goals: Motivation and improvement in self-esteem and mental health promoted through community engagement linked to SMART goals. For example, completing a healthy meal plan×7 days could result in a donation to a local food bank. Finishing a meditation or mindfulness activity as a family for a week could support a mental health organization or school counseling program. Walking a certain distance might lead to a donation to a park or animal shelter.

11. Gamification: The core feature is the gamified daily quests. These quests are tailored based on an initial barrier assessment and fall into categories like psychological factors, physical activity, mental health, nutrition, intuitive eating, anti-bullying, and hope and resilience. The parameters of the quest are defined by a SMART goal. The app also features a daily journal for tracking wellness metrics.

12. Engagement by Customization: Users can spend earned coins in the customization store to personalize their avatar, LUMO the dragon. Future updates will include additional mythical avatars. If users neglect to earn coins and feed LUMO for three days, the dragon will disappear, requiring extra daily tasks to locate and bring LUMO back.

In the preferred embodiment, the APP addresses childhood wellness by addressing the diverse needs of individuals, particularly given the complexity of social, behavioral, and environmental factors that influence health. Social determinants of health (SDOH) such as access to safe environments for physical activity, availability of nutritious food, and emotional well-being must be considered when designing health interventions for children aged 6 to 18. Current solutions are often generalized and fail to account for individual differences or allow users to play an active role in selecting their wellness goals.

The preferred embodiment seeks to address these limitations by offering a personalized, dynamic wellness plan that begins with an assessment of the child's barriers, focusing on SDOH, and evolves into a customized plan that engages users directly in selecting their wellness goals. These plans include age-specific goals and lessons, tailored to three primary age groups (6-8, 9-12, and 13+), ensuring relevance and engagement.

The first step is a barrier assessment. The user, or their parent/guardian, depending on age completes an initial assessment upon joining the platform. This assessment includes questions on physical activity levels, access to nutritious food, emotional well-being, family support, and other SDOH. For younger users, the questions are presented in a simplified, child-friendly format, while older users receive more in-depth questions to accurately assess their health environment. The assessment data is the processed by an algorithm or a trained AI that categorizes the barriers into the following areas: (1) physical activity (e.g., lack of safe spaces to play); (2) Nutrition (e.g., limited access to healthy foods); (3) Emotional well-being (e.g., stress, bullying); and (4) family and social support (e.g., lack of parental involvement in health).

The next step in the barrier assessment is an analysis on the social determinants of health. The App uses the barrier data to prioritize which SDOH are most significantly impacting the user's wellness. For example, if a child reports limited access to safe outdoor spaces, physical activity is identified as a top barrier. Similarly, if emotional issues like anxiety or stress are prominent, the system may highlight psychosocial factors as a key barrier. The assessment is determined from the App's database or a trained AI.

After the barrier assessment the user goal selection selects goals. The system presents the top three identified barriers to the user. Depending on their age, the user or their parent/guardian can review these barriers and select one to focus on. This process is designed to increase user engagement by giving them agency over their wellness journey. Goal selection is accomplished from the App's database or a trained AI.

Once a barrier is selected, the user is placed on a 60-day quest specifically designed to address that barrier. Each quest is structured around developmentally appropriate goals, challenges, and educational content tailored to the user's age group. For example, 6-8 years old, quests are structured as simple, playful activities (e.g., “Adventures in the Park” for physical activity); for 9-12 years old, quests introduce structured goals and peer-related challenges (e.g., “Sports Star” to improve physical activity and teamwork); and for 13+ years old, quests focus on more complex tasks and self-directed goals (e.g., “Wellness Warrior” for emotional well-being and stress management).

As part of the quest, users are guided through the process of setting a SMART goal that directly relates to their chosen barrier. The system offers suggested goals based on age and previous assessment data but also allows the user to customize these goals to their individual preferences. SMART goals can be adjusted throughout the quest as the user makes progress or encounters difficulties.

Throughout the 60-day quest, the system continuously tracks the user's progress, providing positive reinforcement, educational content, and suggestions for improvement. Users receive notifications to adjust their goals if they're falling behind or to push their goals further if they're excelling. This ensures an adaptive, flexible approach to wellness.

Applicant(s) reserves the right to submit claims directed to combinations and subcombinations of the disclosed inventions that are believed to be novel and non-obvious. Inventions embodied in other combinations and subcombinations of features, functions, elements and/or properties may be claimed through amendment of those claims or presentation of new claims in the present application or in a related application. Such amended or new claims, whether they are directed to the same invention or a different invention and whether they are different, broader, narrower or equal in scope to the original claims, are to be considered within the subject matter of the inventions described herein.

1. A method for scaling a weight loss treatment plan by:
(i) providing a mobile application that allows for:
(a) entering the health information of a user;
(c) providing the user with nutritional information;
(d) providing the user with exercise tips;
(e) providing links to web-based sites offering continued education on weight loss and fitness;
(f) gamification and
(ii) where the mobile application further provides:
(a) collecting, tracking, and storing pertinent data;
(b) monitoring the user's adherence to recommendations and tracking of lifestyle changes;
(c) providing feedback and recommendations to the user; and
(d) communicating to a provider said pertinent data, said adherence to recommendations, and progress on said lifestyle changes.

2. The method claim 1, where gamification further includes engagement of a user by including an avatar that interacts with the user.

3. The method of claim 2, where the avatar interacts using a speech bubble, or by performing tricks.

4. The method of claim 1, where the points are earned by completing quests.

5. The method of claim 4, where the quests are in the form of daily lessons.

6. The method of claim 5, where the quests are in the form of multiple-choice questions.

7. The method of claim 1, where the points are provided as virtual coins.

8. The method of claim 7, where the coins are awarded by interacting with a virtual device.

9. The method of claim 1, where the collection of pertinent data is accomplished by journal entries by the user; where journal entries are in the form of answering multiple-choice questions; or are made by selecting an emoji; or are made by entering text.

10. A method for scaling a weight loss treatment plan by:
(i) providing a mobile application that allows for:
(a) entering the health information of a user;
(b) running a user assessment;
(c) gamification; and
(ii) where the mobile application further provides:
(a) collecting, tracking, and storing pertinent data;
(b) monitoring the user's progress, tracking of lifestyle changes, facilitating feedback, providing recommendations, and support to the user.

11. The method of claim 10 where the user assessment further includes an age appropriate barrier assessment.

12. The method of claim 11 where the barrier assessment is analyzed and the user is provided with at least one goal based on the analysis.

13. The method of claim 12 where at least one of the goals is gamified by converting it to an age-appropriate quest.

14. The method of claim 13 where the objectives of the quest are defined as SMART goals.

15. The method of claim 14 gamification further includes awarding points for completing quests.

16. The method of claim 15 were gamification further includes an age appropriate interactive avatar.

17. A method for moving a user through a treatment plan by:
(a) entering a user's heath information;
(b) running a user barrier assessment;
(c) identifying a list of barriers;
(d) based on barriers, defining a prioritized treatment plan in the form of at least one gamified quest;
(e) defining the quest as a SMART goal; and
(f) completing the SMART goal.

18. The method of claim 17 further comprises gamification where gamification comprises:
(a) engagement of a user by including an avatar that interacts with the user;
(b) where the avatar interacts with the user by a speech bubble;
(c) where the avatar interacts with the user by performing tricks;
(d) where the user earns points by completing quests;
(e) where the quests are in the form of daily lessons taught by the avatar;
(f) where the quests are in the form of multiple-choice questions;
(g) where the points are awarded by interacting with a virtual device;
(h) the points can take the form of any one of the following virtual: coins, gold coins, silver coins, currency, gems, food items, diamonds, rubies, sapphires, or marbles.

19. The method of claim 18, where the points are spent to maintain interaction with the avatar.
